SEQUENCE LISTING (11 sequences)

SEQ ID NO: 1 - DNA, 301 bp, Artificial sequence
  synthetic sequence
acataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtg

SEQ ID NO: 2 - DNA, 348 bp, Artificial sequence
  synthetic sequence
tacctatttgcaaaagaagggaaacccccgcgtatggaaccagaagagggtttcttgcagaaggtgccccaaggtgttgcagtttgataacgctctcctcatatctgttaaagagcgcggcaaatgttatctggaagcagccgggacaagagcacccccagttctgagcacagtaactgggctactcagaagttccactgaacggcatgtcagtgccttaccaatagtcacactgagctgcttgcaaagtaactgccaccacattcctttcttaaagtggcctgattccaaagagcttgcctagagcaaggggcagaagagctgccaagtgcctgccatttccaggaa

SEQ ID NO: 3 - DNA, 302 bp, Artificial sequence
  synthetic sequence
ccagtttaccacctaagaataataatagtatcatcttcataggggagttgttgtgactttgagattagctttatcaagtgtttataacagtacctggcacatggctgaatgagtgcaactctgcctgcttttgattgaagtcatattttgtgacagtttctgttgaagactgggcaccaagctagaccacttcatgtgcattttggagataaaagttttatctagcattgcagctgcaaagcccctcctctctctcttcactttgttggtttcctttttatctttacttcggtgtttttcat

SEQ ID NO: 4 - DNA, 301 bp, Artificial sequence
  synthetic sequence
tcccatctatctgccttgcatggtgacagccagcctttagcaacttctaaaagaaagaggatctatcagctcagtctgagcaggcatgataaggtctcctcccttgcctggaggcactcatctgagcctccaaaccaccccattagtgaattctcagaaagaacattctgctgagaaagaggaggataggttcccagtcaaggctgggagcttgtagccactaatctcttggtgtctagatcaggtacaaggctgtctctctgtccccaccctctctggtcccctcctcttactgtccact

SEQ ID NO: 5 - DNA, 374 bp, Artificial sequence
  synthetic sequence
gggtgtgtggctccttaaggtgacccagcagccctgggcacagaagtggtgcgtggagataatgccaacagtgataaccagcagggcctgtcagaagaggccctggacactgaaggctgggcacagccttggggaccgctcacaggacatgcagcagtgtgtgccgacaactccctaccgcgacccctatcagtgccgaccaagcacacaagatgcacacccaggctgggctggacagaggggtcccacaagatcacagggtgtgccctgagaaggtggggagctcacagcctccaagcattgcatcatcctggtaccaggaaggcaatgggctgccccatacccacttcccttcctagaattggcctgggaca

SEQ ID NO: 6 - DNA, 301 bp, Artificial sequence
  synthetic sequence
tccaaggcagcatggcgggcaagaagttgaggccactgtccctgggtgttcctacccccacaccctcaccccaagacagcctgttactgcggcgccaacagccacggtcgcctacatctgataagacttatctgctgccccagggcaggccggagctggcgtaagccccagtggggcgctaagtgagtgtgcccctgcctcccgccagcactggcctggcctgcaggcttagcctgggtcatcaaggtatcccacaggctctagttcaaatccagcagaacctctctgagcctcactcttc

SEQ ID NO: 7 - DNA, 301 bp, Artificial sequence
  synthetic sequence
caacaaaaacaagcaaacaaatagaacctcaggctcacggctgggcaagagagaaagcacacgatgatggacatctggagcttccagcaatgcatgggcagcaaagataagctttacttgactgctgggtaggagcaccagcagaatgaagcatagactatttaccacaccctactttggcttgggctaatagtaagttacgcttgtacaaggtcttggaaagcagccagtgcactggcactctaagcctcacaaaagatagtgcttttcaggtagaaaatatattccacaattggtctct

SEQ ID NO: 8 - DNA, 301 bp, Artificial sequence
  synthetic sequence
gtcccaggtcacacagcttgagggtggcagaagccagatctcaggtcacagagtggctctcctccatttgcctccctggtcttgcctcctggaggcctgtagataggagacacccttcctggaaagacaacagcttggggacattacagataaggctgggcgggtgtgcctagctagcccagaataaaaggatcaccgagagaagttaggaatcgaccttccttgaggtgggttctgcagataaagatgcttttgaagttcagttggtgggagataggatctttgtggccttctggatgga

SEQ ID NO: 9 - DNA, 301 bp, Artificial sequence
  synthetic sequence
ccagtggtttggggacatgcttcctggagtcttatcccctcttgtcagattcaggtttaggacaacggtacagttgttccattgttcccagataacatatcctatcttaacgtctttgctggggcagcccttggacacaccctgagctcctgccctatcactggccccacttgtaccccttgtgggcaggcaccacggcagcctgctctgaagggagctttcatgtggccgaccaactctactccttgactcctcccttctctcattgaaaaaaaatgtttaaggggacatttctctctcc

SEQ ID NO: 10 - DNA, 301 bp, Artificial sequence
  synthetic sequence
gtagaaagggatcctttaagatgaaatcgatgcctcctcatggagttctaagccctcagaagtggaagtcaactggagtgtgcaattttctatctttcaactaacccaacagtttctacctcaccctcctgccttcctaggactctgtgcacacacccaccagagtgttatcacattgcaagacagaacccaattgtctcctttatctcctctaccagactttgtgctccatcagttgaagagtaggatgaagaaaaaaaaaaaaggttacatgaagatgatggaattataatcaggcctt

SEQ ID NO: 11 - DNA, 612 bp, Artificial sequence
  synthetic sequence
  misc_feature (302)..(311): n is a, c, g, or t
gtcccaggtcacacagcttgagggtggcagaagccagatctcaggtcacagagtggctctcctccatttgcctccctggtcttgcctcctggaggcctgtagataggagacacccttcctggaaagacaacagcttggggacattacagataaggctgggcgggtgtgcctagctagcccagaataaaaggatcaccgagagaagttaggaatcgaccttccttgaggtgggttctgcagataaagatgcttttgaagttcagttggtgggagataggatctttgtggccttctggatggannnnnnnnnnccagtggtttggggacatgcttcctggagtcttatcccctcttgtcagattcaggtttaggacaacggtacagttgttccattgttcccagataacatatcctatcttaacgtctttgctggggcagcccttggacacaccctgagctcctgccctatcactggccccacttgtaccccttgtgggcaggcaccacggcagcctgctctgaagggagctttcatgtggccgaccaactctactccttgactcctcccttctctcattgaaaaaaaatgtttaaggggacatttctctctcc

(19) United States
(12) Patent Application Publication
(10) Pub. No.: US 2022/0411822 A1
(43) Pub. Date: Dec. 29, 2022

(54) Novel Erythroid Specific Enhancers and Uses Thereof

(71) Applicant: ALTIUS INSTITUTE FOR BIOMEDICAL SCIENCES, Seattle, WA (US)

(72) Inventors: NIKOLETA PSATHA, Seattle, WA (US); JEFF VIERSTA, Seattle, WA (US); GRIGORIOS GEORGOLOPOULOS, Seattle, WA (US); JOHN A. STAMATOYANNOPOULOS, Seattle, WA (US)

(21) Appl. No.: 17/793,793

(22) PCT Filed: Jan. 22, 2021
(86) PCT No.: PCT/US2021/014752

§ 371 Date: Jul. 19, 2022

Related U.S. Application Data
(60) Provisional application No. 62/964,298, filed on Jan. 22, 2020.

(51) Int. Cl. C12N 15/86; C12N 15/62

(52) U.S. Cl. CPC C12N 15/86; C12N 15/62; C12N 2830/15; C12N 2740/16043; A61K 48/00

(57) ABSTRACT

Provided herein are expression cassettes comprising at least one copy of an enhancer element, wherein the enhancer element comprises or consists essentially of a nucleotide sequence at least 50% identical to any one of SEQ ID NOs: 1-10 and vectors comprising the expression cassettes. Also provided herein are cells transduced with the expression cassettes or the vectors. Further described herein are pharmaceutical compositions comprising an effective amount of one or more of: the cell, the expression cassette, or the vector of this disclosure. Also disclosed herein are methods of treating a hemoglobinopathy in a subject, comprising administering an effective amount of the pharmacological compositions described herein.

### CROSS-REFERENCE TO RELATED APPLICATIONS

This application claims priority from U.S. Application Ser. No. 62/964,298, filed Jan. 22, 2020 which is hereby incorporated by reference in its entirety.

### STATEMENT OF GOVERNMENT SUPPORT

This invention was made with government support under grant number R01HL136375-01 awarded by National Heart, Lung, and Blood Institute (NHLBI). The government has certain rights in the invention.

### INCORPORATION BY REFERENCE OF SEQUENCE LISTING PROVIDED AS TEXT FILE

A Sequence Listing is provided herewith as a text file, “ALTI-728WO_SEQ_LIST_ST25.txt,” created on Jan. 22, 2021 and having a size of 8 KB. The contents of the text file are incorporated by reference herein in their entirety.

### BACKGROUND

β-thalassemia and sickle cell anemia are severe congenital anemias that are caused by defective production of the b chain of hemoglobin. In β-thalassemia, the β chain deficit leads to the intracellular precipitation of excess α-globin chains, causing ineffective erythropoiesis and hemolytic anemia (Weatherall and Clegg (1981), Stamatoyannopoulos et al. (1994), Weatherall (2001), Steinberg (2001)). In sickle cell anemia, the hemoglobin β chain is mutated at amino acid position 6 (Glu Val), leading to the synthesis of β^{S} instead of the normal β^{A} chain (Steinberg (2001), Pauling et al. (1949)). The resulting hemoglobin, HbS, causes accelerated red cell destruction, erythroid hyperplasia and painful vaso-occlusive ‘crises’ (Steinberg (2001)). After almost 20 years of development of globin gene therapy vectors, stem cell gene therapy for the hemoglobinopathies has progressed substantially and several patients have been treated. In non-β⁰/β⁰ patients, transfusion independence has been accomplished. Specifically, for most HbE/βthal patients treated to date the vector-derived beta chain production together with the endogenous beta E and fetal hemoglobin activation can lead to transfusion independence. The results in β⁰ thalassemia patients however are less encouraging, since very few of them have so far became transfusion independent.

To achieve high globin gene expression all currently available vectors use components of the enhancer of the β globin locus, the Locus Control Region (LCR). The function of the LCR is quite complex. It is likely that this element evolved during the almost half a billion years of globin gene evolution not simply because the locus needed an extremely strong enhancer but also because of the requirement for an enhancer regulating the switches of globin genes during development. The five DNase I Hypersensitive sites (DHS) which compose the LCR span a distance of 20 kb and the question has been how to reduce the LCR to a size manageable in the viral vectors and keep at least part of its activity. The sizes of the micro-LCRs (μLCRs) of the vectors currently used in clinical trials range from 2.7 to 3.4 kb. Importantly, the large size of these μLCR enhancers adversely affects the titers of the globin gene lentiviral vectors. Low titers are among the reasons for the lower stem cell transduction efficiencies of the globin vectors compared to the enzymopathy or immunodeficiency vectors.

In addition to low efficiency there are several concerns regarding these vectors' safety, since the traditional view that the beta-globin LCR is required for the safety of globin gene vectors because it is erythroid lineage specific has been proven as incorrect. First, the μLCR containing lentiviral vectors activate flanking genes in in vitro expanded early hematopoietic cells. Second, the stronger enhancer of the LCR, DHS2, is ubiquitously present in all hematopoietic cell lines and lineages and is also present in the undifferentiated human embryonic stem cells. Third, there is evidence that the LCR is active in multipotent and in bipotent hematopoietic progenitors. Fourth, evidence that the LCR is active in earlier stages of hematopoiesis also comes from the analysis of the activation of the cellular growth gene that led to the clonal stem cell expansion in the first thalassemia patient treated with gene therapy; of the 10,000 fold activation of that gene, the 700 fold activation was due to the μLCR enhancer contained in the lentiviral vector, an event that occurred in a multipotent myeloid progenitor.

Furthermore, even though μLCR is a strong and potent enhancer for all the globin gene addition vectors, its strength could be a drawback in novel lentiviral vectors for hemoglobinopathies, such as shRNA vectors for HbF repressors, i.e., the BCL11a shRNA vector. It has been shown that massive overloading of cells with exogenous RNAi inducers can induce competition for the endogenous miRNA machinery leading to substantial cell toxicity. Therefore, for these vectors a more moderate enhancer would be ideal.

### SUMMARY

Provided herein are expression cassettes comprising at least one copy of an enhancer element, wherein the enhancer element comprises or consists essentially of a nucleotide sequence at least 50% identical to any one of SEQ ID NOs: 1-10 and vectors comprising the same. Also provided herein are cells transduced with said expression cassettes. In some aspects, described herein are cells transduced with said vectors. Further described herein are pharmaceutical compositions comprising an effective amount of one or more of: the cell, the expression cassette or the vector of this disclosure. Also disclosed herein are methods of treating a hemoglobinopathy in a subject, comprising administering an effective amount of the pharmacological composition described herein.

### BRIEF DESCRIPTION OF THE FIGURES

FIG. 1 shows strong transgene expression using vectors comprising novel enhancer elements. Transgene expression in human erythroid cells line employing the new, short (300 bp) erythroid enhancers was compared to the entire (>800 bp) beta globin HS2 enhancer element. Transduction was performed in various MOIs (Multiplicity of Infection). The range of expression is depicted as a box plot. The majority of the novel vectors enhanced transgene expression significantly over the HS2 enhancement levels. Vectors L070 and L077 include enhancers having sequences set forth in SEQ ID NO: 1 and SEQ ID NO: 2, respectively.

FIG. 2 shows erythroid specific transgene expression using the new vectors comprising enhancer elements disclosed herein. All the new vectors have erythroid specific expression profiles. CD34+ cells were transduced with all lentiviral vectors depicted in FIG. 1 and were subsequently differentiated towards the erythroid, granulocytic/monocytic and megakaryocytic lineage. In parallel T-cells were transduced with similar virus volumes to the CD34 transductions. Minimal to no transgene expression was observed in all non-erythroid cell lines. The vector with the μLCR enhancer directed the highest expression levels in all non-erythroid lineages.

FIG. 3 provides a schematic of a therapeutic vector. The therapeutic gene is operably linked to an erythroid promoter and an enhancer, which is operably linked to a CMV promoter/enhancer element. Viral transcription is driven by the CMV promoter.

FIG. 4 provides a schematic depiction of the reporter vector used to identify novel erythroid specific enhancers. This reporter vector is a second generation lentiviral vector insulated with the C1 insulator. Transcription of the vector is driven by the endogenous LTR.

FIG. 5 shows that novel vectors show a more than two fold increase in transducibility as compared to a no enhancer vector and to the μLCR enhancer vector currently used in many clinical vectors. All vectors were produced in parallel. Vectors L070 and L077 include enhancers having sequences set forth in SEQ ID NO: 1 and SEQ ID NO: 2, respectively.

FIG. 6 provides a comparison of CD34+ cell transducibility between the μLCR-T87Q-beta globin vector and the L070-T87Q-beta globin vector, an overall two-fold increase of the number of transduced cells is observed using the latter. Furthermore, almost a three-fold increase of the cells with more than 5 integrated vector copies was observed, which represents the population that attributes most to a therapeutic outcome.

FIG. 7 shows that combining two of the novel enhancers a 600 bp enhancer element was created. This synthetic short enhancer achieves transgene expression levels similar to the 2.7 kb LCR enhancer of the currently used clinical vectors.

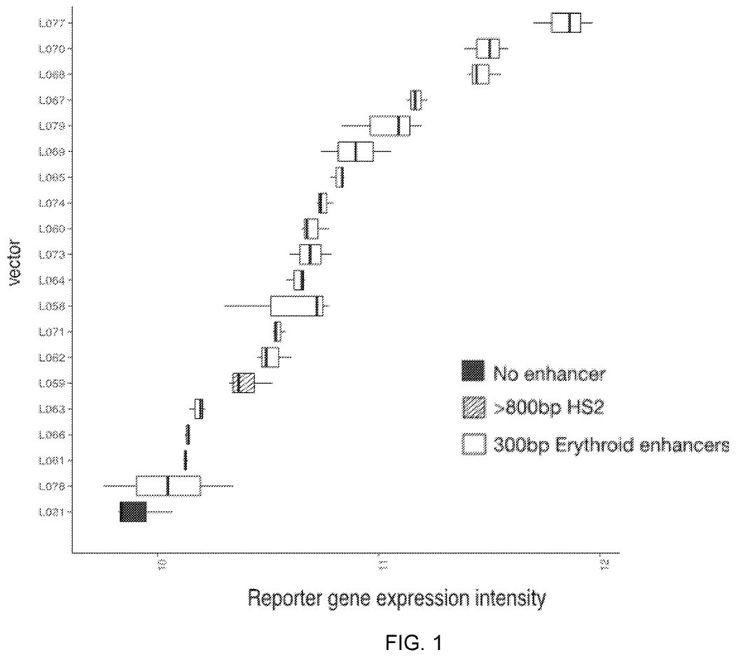

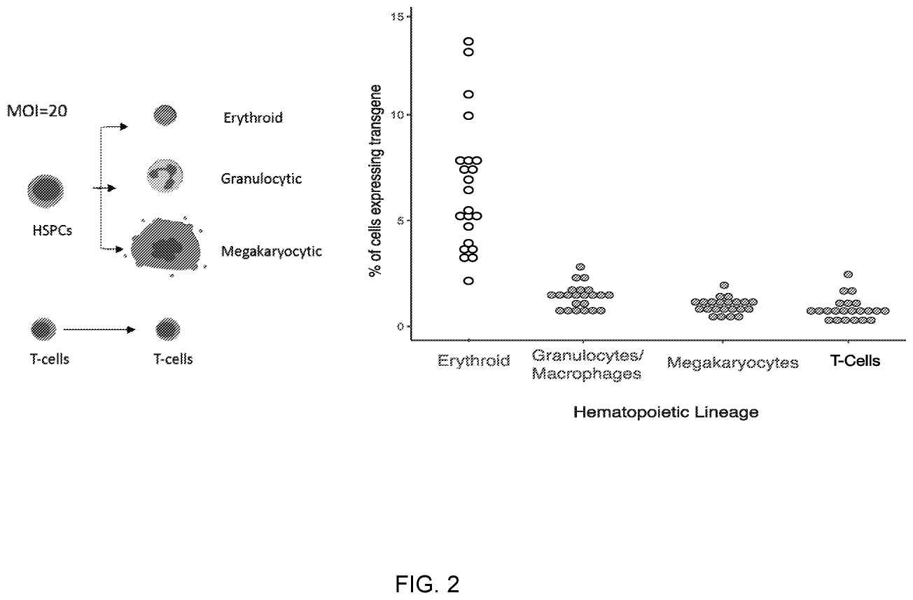

### DETAILED DESCRIPTION

Before the methods of the present disclosure are described in greater detail, it is to be understood that the methods are not limited to particular aspects described, as such may, of course, vary. It is also to be understood that the terminology used herein is for the purpose of describing particular aspects only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate aspects, may also be provided in combination in a single aspect. Conversely, various features of the methods, which are, for brevity, described in the context of a single aspect, may also be provided separately or in any suitable sub-combination. All combinations of the aspects are specifically embraced by the present disclosure and are disclosed herein just as if each and every combination was individually and explicitly disclosed, to the extent that such combinations embrace operable processes and/or compositions. In addition, all sub-combinations listed in the aspects describing such variables are also specifically embraced by the present methods and are disclosed herein just as if each and every such sub-combination was individually and explicitly disclosed herein.

As will be apparent to those of skill in the art upon reading this disclosure, each of the individual aspects described and illustrated herein has discrete components and features which may be readily separated from or combined with the features of any of the other several aspects without departing from the scope or spirit of the present methods. Any recited method can be carried out in the order of events recited or in any other order that is logically possible.

### Definitions

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Aspects defined by each of these transition terms are within the scope of this disclosure.

As used herein, the term “expression” refers to the process by which a polynucleotide is transcribed from a DNA template (such as into a mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “β-globin locus control region (LCR)” refers to a polynucleotide composed of one or more Dnase I hypersensitive site (DHS or HS) regions, including a HS1 region, a HS2 region, a HS3 region, and a HS4 region. The structure of many LCRs of the β-globin genes have been published, e.g., human (Li et al, J. Biol. Chem. (1985); 260: 14,901; Li et al, Proc. Natl Acad. Sci. (1990) 87:8207); mouse (Shehee et al, J. Mol. Biol. (I989); 205:41); rabbit (Margot et al., J. Mol. Biol. (1989); 205: 15); and goat (Li, Q., et al., Genomics (I99I); 9:488), each of which are incorporated by reference herein.

As used herein, the term “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. “Heterologous” in the context of recombinant cells can refer to the presence of a nucleic acid (or gene product, such as a polypeptide) that is of a different genetic origin than the host cell in which it is present.

“Heterologous” in the context of a polynucleotide can include operably linked nucleic acid sequences that are derived from different sources, e.g., different organisms, different gene, etc. Exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin relative to the promoter, the coding sequence or both).

As used herein, the term “globin” refers to a family of heme-containing proteins that are involved in the binding and transport of oxygen. Subunits of vertebrate and invertebrate hemoglobins, vertebrate and invertebrate myoglobins or mutants thereof are included by the term globin.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene region, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide can comprise one or more modified nucleotides, such as methylated nucleotides and nucleotide analogs. In particular aspects, the presently disclosed subject matter provides polynucleotides encoding one or more globin genes or functional portions thereof. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the terms “polypeptide” and “protein” are used interchangeably to refer to a polymer of amino acid residues and to synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. Particular aspects of the presently disclosed subject matter also include polypeptides that are distinguished from a reference polypeptide by the addition, deletion, truncations, and/or substitution of at least one amino acid residue, and that retain a biological activity. In certain aspects, the polypeptide is distinguished from a reference polypeptide by one or more substitutions, which may be conservative or non-conservative, as known in the art. In certain aspects, the polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain aspects, the amino acid additions or deletions occur at the C-terminal end and/or the N-terminal end of the reference polypeptide. In certain aspects, the amino acid deletions include C-terminal truncations of about 1, about 2, about 3, about 4, about 5, about 6, about 7, about 8, about 9, about 10, about 15, about 20, about 25, about 30, about 35, about 40, about 45, 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, or about 175 or more amino acids, including all intervening numbers of amino acids, e.g., 25, 26, 27, 29, 30 . . . 100, 101, 102, 103, 104, 105 . . . 170, 171, 172, 173, 174, etc.

As noted above, polypeptides of the presently disclosed subject matter may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

As used herein, the term “promoter” refers to a region of a polynucleotide (DNA or RNA) to which an RNA polymerase binds and facilitates transcription from a coding sequence operably linked thereto. The terms “enhancer” and “enhancer element” refer to a segment of DNA which contains sequences capable of providing enhanced transcription and in some instances can function independent of their orientation relative to the promoter. An enhancer can function cooperatively or additively with promoters and/or other enhancer elements. A promoter or enhancer is considered erythroid lineage-specific or erythroid specific when it provides for expression of a nucleotide coding sequence of interest only in a cell of the erythroid lineage. Cells of the erythroid lineage include, but are not limited to, e.g., proerythroblasts, basophilic erythroblasts, polychromatophilic erythroblasts, orthochromatic erythroblasts, polychromatophilic erythrocytes, and erythrocytes (red blood cells).

As used herein, the term “expression cassette” refers to a nucleic acid construct, generated recombinantly or synthetically, with a series of specified nucleic acid elements, which permit transcription of a particular nucleic acid in a target cell. The expression cassette can be incorporated into a plasmid, chromosome, mitochondrial DNA, plastid DNA, virus or nucleic acid region. The expression cassette can include a gene to be transcribed and elements that control the expression of the gene (e.g., a promoter for driving transcription of the gene and an enhancer to regulate the promoter for e.g., enhancing transcription of the gene). In some aspects, the expression cassette includes the coding sequence of a therapeutic agent used to treat, prevent, or ameliorate a genetic disorder. Such a genetic disorder may be a hematopoietic disorder and the coding sequence of the therapeutic agent may be operably linked to an erythroid specific enhancer and promoter. The cassette may further include other DNA sequences, such as untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, nucleotides encoding self-cleaving polypeptides or epitope tags.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vectors, as well as viral vectors and plasmids.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. gRNA comprises or alternatively consists essentially of, or yet further consists of a fusion polynucleotide comprising CRISPR RNA (crRNA) and trans activating CRIPSPR RNA (tracrRNA); or a polynucleotide comprising CRISPR RNA (crRNA) and trans-activating CRIPSPR RNA (tracrRNA). In some aspects, the gRNA is synthetic (Kelley, M. et al. (2016) J of Biotechnology 233 (2016) 74-83).

The term “Cas9” refers to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida and Cpfl (which performs cutting functions analogous to Cas9) from various bacterial species including Acidaminococcus spp. and Francisella novicida JJ112.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which may be a wild-type or mutated FokI endonuclease.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN can include a FokI nuclease domain (or derivative thereof) fused to a DNA-binding domain. In the case of a ZFN, the DNA-binding domain comprises one or more zinc fingers. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, reduction in the number of disease episodes and/or symptoms, amelioration or palliation of the condition (including disease), increase in the length of disease-free presentation following treatment, and/or decreased mortality at a given point of time following treatment, whether detectable or undetectable. Treatments containing the disclosed compositions and methods can be used as a sole therapy or in combination with other appropriate therapies. According to some aspects, treatment excludes prophylaxis.

An “effective amount” or “efficacious amount” is an amount sufficient to achieve the intended purpose. According to some aspects, the effective amount is one that functions to achieve a stated therapeutic purpose, e.g., a therapeutically effective amount. As described herein in detail, the effective amount, or dosage, depends on the purpose and the composition, and can be determined according to the present disclosure.

As used herein, the term “administer” and “administering” are used to mean introducing the therapeutic agent into a subject. The therapeutic administration of this substance serves to attenuate any symptom or prevent additional symptoms from arising. When administration is for the purposes of preventing or reducing the likelihood of developing an autoimmune disease or disorder, the substance is provided in advance of any visible or detectable symptom. Routes of administration include, but are not limited to, oral (such as a tablet, capsule or suspension), parenteral, topical, transdermal, intranasal, subcutaneous, intravenous, intraarterial, intramuscular, intraosseous, intraperitoneal, epidural, and intrathecal.

As used herein, the term “hemoglobinopathy” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins is present in the blood (e.g., sickle cell/Hb-C disease).

The term “sickle cell anemia” or “sickle cell disease” is defined herein to include any symptomatic anemic condition which results from sickling of red blood cells. Manifestations of sickle cell disease include, e.g., anemia, pain, and/or organ dysfunction such as renal failure, retinopathy, acute-chest syndrome, ischemia, priapism and stroke. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS.

As used herein, the term “thalassemia” encompasses hereditary anemias that occur due to mutations affecting the synthesis of hemoglobin. Thus, the term includes any symptomatic anemia resulting from thalassemic conditions such as severe or β thalassemia, thalassemia major, thalassemia intermedia, and thalassemias such as hemoglobin H disease. A thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles.

As used herein, the term “autologous,” in reference to cells, tissue, and/or grafts refers to cells, tissue, and/or grafts that are isolated from and then and administered back into the same subject, patient, recipient, and/or host. “Allogeneic” refers to non-autologous cells, tissue, and/or grafts.

The term “subject,” “host,” “individual,” and “patient” are used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some aspects a mammal is a human. A mammal can be any age or at any stage of development (e.g., an adult, teen, child, infant, or a mammal in utero). A mammal can be male or female. A mammal can be a pregnant female. In some aspects, a subject is a human.

A “composition” refers to a combination of the active agent and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffmose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

### Expression Cassettes

Provided herein is an expression cassette comprising an enhancer element that is shorter than the currently used enhancers for enhancing gene expression, such as, the beta-globin locus control region, μLCR, and β-globin DHS2. The shorter length of these enhancers improves production of nucleic acids such as expression cassettes and vectors, providing in many cases improved productions of vectors at a lower cost and further increasing the specificity of expression of the therapeutic gene. As used herein, the phrase “therapeutic gene” refers to a nucleic acid sequence that encodes a therapeutic agent, such as, a RNA or a polypeptide.

In certain aspects, the enhancer element comprises, consists essentially of, or consists of a nucleotide sequence at least about 50% identical to any one of SEQ ID NOs: 1-10. In certain aspects, the nucleotide sequence of the enhancer element is at least about 50%, at least about 60%, at least about 70%, at least about 80%, at least about 85%, at least about 90%, at least about 95%, at least about 96%, at least about 97%, at least about 98%, at least about 99%, at least about 99.5% or about 100% identical to any one of SEQ ID NOs: 1-10.

In certain aspects, the enhancer element is at least 200 bp long and up to 1 kb long. In certain aspects, the enhancer element is up to 900 bp, up to 875 bp, up to 800 bp, up to 700 bp, up to 600 bp, up to 500 bp, up to 400 bp, or up to 350 bp long. In certain aspects, the enhancer element is 250 bp-900 bp, 250 bp-875 bp, 250 bp-800 bp, 250 bp-700 bp, 250 bp-600 bp, 250 bp-500 bp, 250 bp-400 bp, 250 bp-350 bp, 275 bp-600 bp, or 300 bp-500 bp in length and has a nucleotide sequence at least 50% identical (e.g., at least 60%, at least 70%, at least 80%, at least 85%, at least 90%, at least 95%, at least 96%, at least 97%, at least 98%, at least 99%, at least 99.5% or 100% identical) to any one of SEQ ID NOs: 1-10. In certain aspects, the enhancer element consists of the nucleotide sequence set forth in any one of SEQ ID NOs: 1-10. In certain instances, the enhancer element comprises a nucleotide sequence that differs from the sequence of any one of SEQ ID Nos:1-10 in length, e.g., the nucleotide sequence is shorter than the sequence of any one of SEQ ID Nos:1-10 by up to 100 bp, up to 80 bp, up to 70 bp, up to 60 bp, or up to 50 bp. In certain instances, the enhancer element comprises a nucleotide sequence that is longer than the sequence of any one of SEQ ID Nos:1-10 by, e.g., up to 100 bp, up to 80 bp, up to 70 bp, up to 60 bp, or up to 50 bp. In addition or alternatively, the enhancer element comprises a nucleotide sequence that is different from the nucleotide sequence set forth in any one of SEQ ID NOs: 1-10 in nucleotide sequence, e.g., the nucleotide sequence may have naturally occurring genomic sequence variation such as deletions, insertions, and/or substitutions that may be found in certain individuals.

In certain aspects, the enhancer element comprises an erythroid transcription factor binding site. In some aspects, the enhancer element comprises an erythroid transcription factor binding site selected from the group consisting of GATA1, TAL1 and KLF1.

According to certain aspects, the expression cassette comprises multiple enhancer elements of the present disclosure. For example, the expression cassette can include two, three, four, five, or more enhancer elements. In some aspects, the expression cassette comprises two copies of the same enhancer element. In certain aspects, the expression cassette comprises a first enhancer element and a second enhancer element having different sequences.

In certain aspects, the expression cassette comprises a promoter which is regulated by the enhancer element to increase transcription of a gene operably linked to the promoter. Promoters of interest include ubiquitous promoters as well as tissue specific promoter, e.g., a hematopoietic lineage specific promoter, such an erythroid specific promoter. In certain aspects, one of more enhancers of the present disclosure are operably linked to a beta-globin promoter.

The expression cassette may further comprise a sequence encoding a therapeutic agent. The sequence encoding a therapeutic agent may be a globin gene, e.g., a β-globin, a γ-globin, or a δ-globin gene. The globin gene may be operably linked to a human erythroid promoter. The globin gene may be a wild type human β-globin gene, a deleted human β-globin gene comprising one or more deletions of intron sequences, or a mutated human β-globin gene encoding at least one anti-sickling amino acid residue. See U.S. Pat. Nos. 6,051,402; 5,861,488; 6,670,323; 5,864,029; and 5,877,288, which are herein incorporated by reference. In some aspects, the expression cassette does not include a β-globin LCR. The enhancers disclosed herein can be positioned at the 3′ UTR (downstream) or the 5′ UTR (downstream) of the sequence encoding a therapeutic agent. In certain aspects, the enhancer disclosed herein is positioned in the 5′ UTR of the β-globin gene. In certain aspects, the enhancer disclosed herein provide erythroid-specific expression of the therapeutic agent.

In some aspects, the therapeutic agent comprises a polynucleotide or a polypeptide. In certain aspects, the therapeutic agent comprises a globin polypeptide, wherein the globin polypeptide is a β-globin, a γ-globin, or a δ-globin. β-globin, γ-globin, and δ-globin may be wild type or mutated. In certain aspects, the therapeutic agent is an shRNA targeting an HbF repressor, a homing endonuclease or a DNA binding protein that reactivates endogenous HbF expression. In some aspects, an shRNA targeting an HbF repressor targets B-cell lymphoma/leukemia 11A (BCL11a), Leukemia/lymphoma-related factor (LRF), heme-regulated inhibitor HRI (also known as EIF2AK1) or Nuclear Factor I X (NFIX). shRNAs targeting HbF repressors are well known in the art, non-limiting examples of such shRNA are described in Guda et al. Mol Ther. 2015 September; 23(9): 1465-1474, Brendal et al. J Clin Invest. 2016 Oct. 3; 126(10): 3868-3878 and Finotti et al. J Blood Med. 2015; 6: 69-85. Homing endonucleases or DNA binding proteins that reactivate endogenous HbF expression are also well known in the art. In some aspects, the expression cassette may mediate forced chromatin looping of a globin gene to its endogenous LCR. In some aspects, the expression cassette may be Ldb1SA-ZFN cassette reported in Cell, 2014 by Deng et al. In certain aspects, the therapeutic agent may be an agent for reducing expression of α-globin. For example, the therapeutic agent for reducing expression of α-globin may comprise shRNA targeting α-globin gene or an endonuclease targeted to the coding or enhancer region of α-globin gene.

### Vectors

Further provided are vectors comprising any of the nucleic acids, such as the expression cassettes of the present disclosure. In some aspects, the vector is a retroviral vector or a lentiviral vector.

In certain aspects, the vector further comprises a reporter or a selectable marker. As is conventional in the art, a reporter or selectable marker is a protein/enzyme whose expression allows identification of cells that have been transformed with a DNA construct or vector containing the gene encoding the protein/enzyme. Selectable markers may provide resistance to toxic compounds such as antibiotics or herbicides, or provide detectable signals such as color or light. Because the genetic code is degenerate, there are many nucleotide sequences that may encode the therapeutic agents of the present disclosure. Polynucleotides that vary due to differences in codon usage are specifically contemplated in particular aspects, for example polynucleotides that are optimized for human and/or primate codon selection.

In certain aspects, the vector further comprises one or more, two or more, three or more, four or more, five or more, or all six of the following:

(a) a restriction enzyme site,

(b) an untranslated region,

(c) a DNase I-hypersensitive site,

(d) a multiple cloning site,

(e) a long terminal repeat, and

(f) a sequence encoding a poly A tail.

According to certain aspects, the vector comprises a first enhancer element, a promoter, a therapeutic gene, and a second enhancer element. In some aspects, the first and second enhancer elements are the same enhancer element. In certain aspects, the first and second enhancer elements are different enhancer elements. According to some aspects, the first and second enhancer elements are positioned in the vector such that they flank the sequence of the promoter and the therapeutic gene.

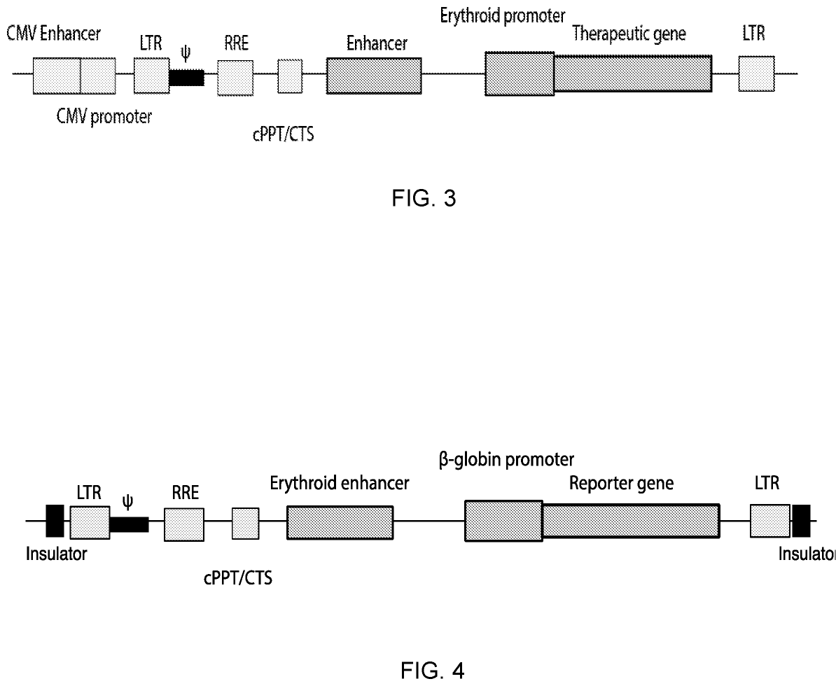

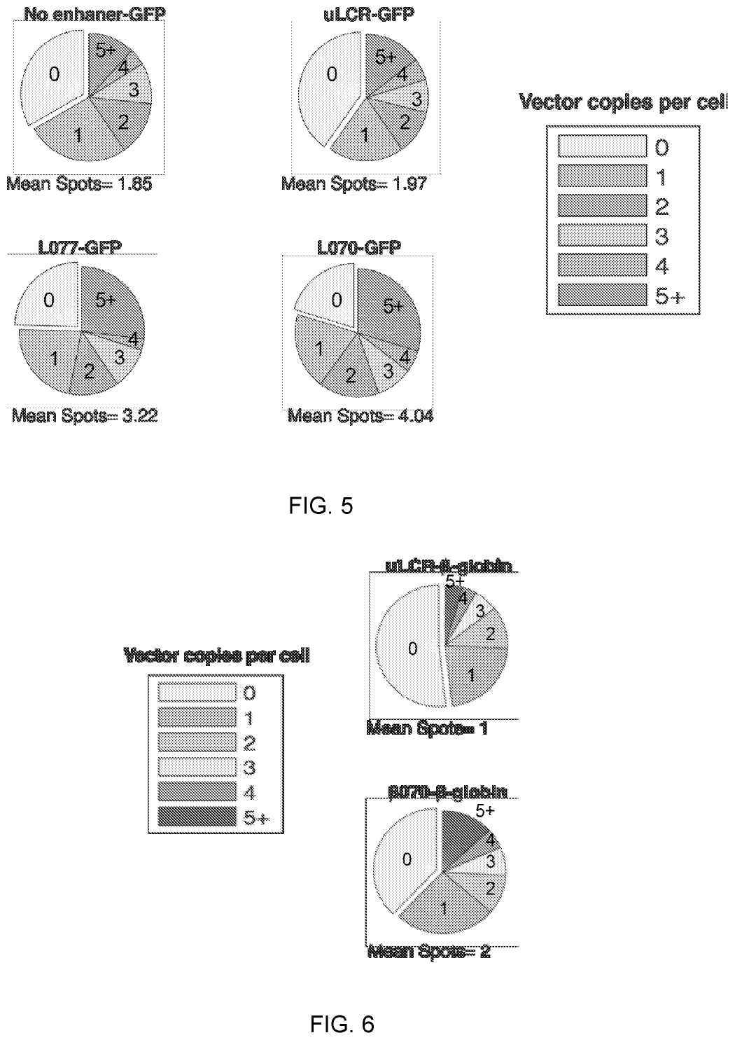

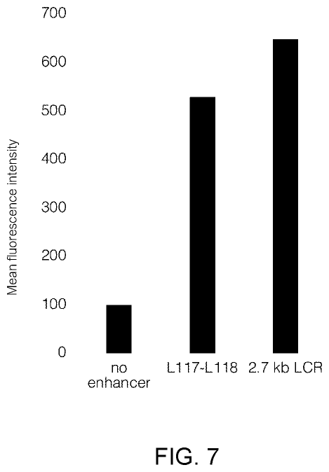

In certain aspects, the vector may be a viral vector and may include a promoter for directing transcription in a virus. In an exemplary aspect, as depicted in FIG. 3, the viral vector comprises a therapeutic gene such as a globin gene operably linked to an erythroid promoter and an enhancer element. The viral vector includes additional components for production of the vector in a virus, including, a promoter such as a CMV promoter. Other promoters may also be utilized for viral transcription and can include, e. g., Rous sarcoma virus (RSV) promoter, simian virus 40 (SV40) promoter, or mammalian elongation factor 1a (EF1α) promoter.

In order to express the expression cassette can be inserted into a vector, e.g., using recombinant DNA techniques known in the art. Exemplary viral vectors include, without limitation, retrovirus (including lentivirus), adenovirus, adeno-associated virus, herpesvirus (e.g., herpes simplex virus), poxvirus, papillomavirus, and papovavirus (e.g., SV40).

Illustrative examples of expression vectors include, but are not limited to, pCIneo vectors (Promega) for expression in mammalian cells, pLenti4/V 5-DEST™, pLenti6/V 5-DEST™ murine stem cell virus (MSCV), MSGV, moloney murine leukemia virus (MMLV), and pLenti6.2/V5-GW/lacZ (Invitrogen) for lentivirus-mediated gene transfer and expression in mammalian cells. In certain aspects, the expression cassettes of the present disclosure may be ligated into any such expression vector for expression in a mammalian cell.

Expression control sequences, control elements, or regulatory sequences present in an expression vector are those non-translated regions of the vector—e.g., origins of replication, selection cassettes, promoters, enhancers, translation initiation signals (Shine Dalgarno sequence or Kozak sequence), introns, a polyadenylation sequence, 5′ and 3′ untranslated regions, and/or the like—which interact with host cellular proteins to carry out transcription and translation. Such elements may vary in their strength and specificity, and can be selected by one skilled in the art depending on the vector system and host to be used for each particular construct. Depending on the vector system and host utilized, any number of suitable transcription and translation elements, including ubiquitous promoters and inducible promoters may be used.

Components of the expression vector are operably linked such that they are in a relationship permitting them to function in their intended manner. In some aspects, the term refers to a functional linkage between a nucleic acid expression control sequence (such as a promoter, and/or enhancer) and a second polynucleotide sequence, e.g., a nucleic acid encoding a globin peptide, where the expression control sequence directs transcription of the globin gene.

In some aspects, the expression vector is an episomal vector or a vector that is maintained extrachromosomally. As used herein, the term “episomal” refers to a vector that is able to replicate without integration into the host cell's chromosomal DNA and without gradual loss from a dividing host cell also meaning that the vector replicates extrachromosomally or episomally. Such a vector may be engineered to harbor the sequence coding for the origin of DNA replication or “ori” from an alpha, beta, or gamma herpesvirus, an adenovirus, SV40, a bovine papilloma virus, a yeast, or the like. The host cell may include a viral replication transactivator protein that activates the replication. Alpha herpes viruses have a relatively short reproductive cycle, variable host range, efficiently destroy infected cells and establish latent infections primarily in sensory ganglia. Illustrative examples of alpha herpes viruses include HSV 1, HSV 2, and VZV. Beta herpesviruses have long reproductive cycles and a restricted host range. Infected cells often enlarge. Non-limiting examples of beta herpes viruses include CMV, HHV-6 and HHV-7. Gamma-herpesviruses are specific for either T or B lymphocytes, and latency is often demonstrated in lymphoid tissue. Illustrative examples of gamma herpes viruses include EBV and HHV-8.

Other gene delivery systems which may be used include mRNA electroporation, CRISPR-Cas9, TALENs, zinc fingers, transposase vectors, and the like. See, e.g., Labanieh et al. (2018) Nature Biomedical Engineering 2:377-391. Further provided herein is an engineered nuclease system comprising the expression cassette of this disclosure. According to some aspects, the nuclease system is an engineered zinc-finger nuclease (ZFN) system, an engineered meganuclease system, or an engineered transcription activator-like effector nuclease (TALEN) system. Also described herein are polynucleotides encoding the nuclease system of this disclosure. In certain aspects, the vector is a lentiviral vector.

Also provided herein is an engineered CRISPR-Cas system comprising the expression cassette of this disclosure. According to some aspects, the CRISPR-Cas system comprises a CRISPR-Cas nuclease and a second enhancer element single-guide RNA. Also disclosed herein are a polynucleotide encoding the CRISPR-Cas system and a vector comprising said polynucleotide. In some aspects, the vector is a lentiviral vector.

### Cells

Aspects of the present disclosure include a cell transduced with the expression cassette described herein. Also provided herein is a cell transduced with the vector of this disclosure. Further provided herein is a cell transduced with the engineered nuclease system of this disclosure.

According to some aspects, the cell is autologous to a subject. In some aspects, the cell is allogeneic. In certain aspects, the cells are stem cells, progenitor cells, or differentiated cells. In certain aspects, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells or hematopoietic stem cells.

In some aspects, the subject is a human. In certain aspects, the cell is selected from the group consisting of a hematopoietic stem cell, an embryonic stem cell, an induced pluripotent stem cell, and a hemogenic endothelium cell. According to certain aspects, the hematopoietic stem cell is a CD34+ hematopoietic stem cell. In some aspects, the cell is transduced ex vivo.

In some aspects, the cells are eukaryotic cells. Eukaryotic cells of interest include, but are not limited to, yeast cells, insect cells, mammalian cells, and the like. Mammalian cells of interest include, e.g., murine cells, non-human primate cells, human cells, and the like.

The terms “recombinant host cells,” “host cells,” “cells,” “cell lines,” “cell cultures,” and other such terms refer to cells which can be, or have been, used as recipients for a recombinant vector or other transferred DNA, and include the progeny of the cell which has been transfected. Host cells may be cultured as unicellular or multicellular entities (e.g., tissue, organs, or organoids) including an expression vector of the present disclosure.

Further provided herein is a population of cells described herein. Also provided are methods of making the cells of the present disclosure. In some aspects, such methods include transfecting or transducing cells with a nucleic acid, expression cassette or vector of the present disclosure. The term “transfection” or “transduction” is used to refer to the introduction of foreign DNA into a cell. A cell has been “transfected” when exogenous DNA has been introduced inside the cell membrane. A number of transfection techniques are generally known in the art. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material. In some aspects, a cell of the present disclosure is produced by transfecting the cell with a viral vector encoding the therapeutic agent of interest.

In some aspects, conditions appropriate for cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of cells.

In some aspects, the nucleic acid is introduced into the cell by microinjection, transfection, lipofection, heat-shock, electroporation, transduction, gene gun, microinjection, DEAE-dextran-mediated transfer, and the like. In some aspects, the nucleic acid is introduced into the cell by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some aspects, the AAV vector comprises ITRs from AAV2 and a serotype from AAV6. In some aspects, the nucleic acid is introduced into the cell by lentiviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one aspect, IDLV vectors including an HIV-based vector backbone (i.e., HIV cis-acting sequence elements) are employed.

### Compositions

Aspects of this disclosure relate to a composition comprising one or more of: the nucleic acid, expression cassette, vector, nuclease system and/or cell of this disclosure. Further provided herein is a pharmaceutical composition comprising an effective amount of one or more of: the cell, the expression cassette, the vector, the nucleic acid, the nuclease system; and a pharmaceutically acceptable carrier. In certain aspects, the composition may be delivered to a subject by use of liposomes, nanocapsules, microparticles, microspheres, lipid particles, or vesicles. The formulation and use of such delivery vehicles can be carried out using known and conventional techniques. It will also be understood that, if desired, the compositions of the invention may be administered in combination with other agents as well, such as, e.g., cells, other proteins or polypeptides or various pharmaceutically-active agents.

In some aspects, the compositions include any of the cell, the expression cassette, the vector, the nuclease system or the nucleic acid of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

In certain aspects, the carrier is a pharmaceutically acceptable carrier. The pharmaceutical compositions generally include a therapeutically effective amount of the cell, the expression cassette, the vector, the nuclease system and/or the nucleic acid. An effective amount can be administered in one or more administrations. The cell, the expression cassette, the vector, the nuclease system or the nucleic acid of the present disclosure can be incorporated into a variety of formulations for therapeutic administration. More particularly, the cell, the expression cassette, the vector, the nuclease system or the nucleic acid of the present disclosure can be formulated into pharmaceutical compositions by combination with appropriate, pharmaceutically acceptable excipients or diluents.

Formulations of the pharmaceutical composition suitable for administration to a patient (e.g., suitable for human administration) are generally sterile and may further be free of detectable pyrogens or other contaminants contraindicated for administration to a patient according to a selected route of administration. The pharmaceutical composition may be formulated for parenteral (e.g., intravenous, intra-arterial, intraosseous, intramuscular, intracerebral, intracerebroventricular, intrathecal, subcutaneous, etc.) administration, or any other suitable route of administration.

Pharmaceutical compositions of the present disclosure may be prepared by mixing the cells, expression cassettes, vectors, nuclease systems or nucleic acids having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid, and/or non-ionic surfactants such as Tween, Brij Pluronics, Triton-X, or polyethylene glycol (PEG).

An aqueous formulation of the cells, expression cassettes, vectors, nuclease systems or nucleic acids of this disclosure may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some aspects, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS).

Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

In some aspects, the pharmaceutical composition includes the cells, expression cassettes, vectors, nuclease systems or nucleic acids of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other aspects, a preservative is included in the formulation, e.g., at concentrations ranging from about 0.001 to about 2% (w/v).

In certain aspects, provided is a pharmaceutical composition that includes a therapeutically effective amount of the cells, expression cassettes, nuclease systems, vectors or the nucleic acids of the present disclosure. A “therapeutically effective amount” of such cells may vary according to factors such as the disease state, age, sex, and weight of the individual, and the ability of the cells to elicit a desired response in the individual. A therapeutically effective amount is also one in which any toxic or detrimental effects are outweighed by the therapeutically beneficial effects. The term “therapeutically effective amount” includes an amount that is effective to “treat” an individual, e.g., a patient. When a therapeutic amount is indicated, the precise amount of the compositions contemplated in some aspects, to be administered, can be determined by a physician in view of the specification and with consideration of individual differences in age, weight, and condition of the patient (individual).

### Methods of Treatment

Aspects of the present disclosure include a method of treating a hemoglobinopathy in a subject, comprising administering an effective amount of the pharmacological composition, cell, expression cassette, vector, nuclease system or nucleic acid of the present disclosure to the subject. According to some aspects, the hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In some aspects, the hemoglobinopathy is β-thalassemia. In some aspects, the hemoglobinopathy is sickle cell anemia. According to some aspects, the subject is a human. In some aspects, the cell comprised in the pharmaceutical composition is from the subject. In certain aspects, the cell is from bone marrow of the subject.

In various non-limiting aspects, vectors or other delivery systems (e.g., nucleases or CRISPR-Cas systems) comprising a presently disclosed expression cassette are administered by direct injection to a cell, tissue, or organ of a subject in need of gene therapy, in vivo. In certain aspects, cells are transduced in vitro or ex vivo with vectors or other delivery systems (e.g., nucleases or CRISPR-Cas systems) of the presently disclosed subject matter, and optionally expanded ex vivo. The transduced cells are then administered to a subject in need of gene therapy, e.g., within a pharmaceutical formulation disclosed herein.

The presently disclosed subject matter provides a method of providing a transduced cell to a subject. In various non-limiting aspects, the method comprises administering (e.g., parenterally) one or more cells (a population of cells) transduced with a presently disclosed expression cassette or a vector or another delivery system (e.g., a nuclease or CRISPR-Cas system) comprising such expression cassette to the subject.

In certain aspects, following administration of one or more of the presently disclosed pharmacological composition, cell, expression cassette, vector, nuclease system or nucleic acid of the present disclosure, peripheral blood of the subject is collected and hemoglobin levels is measured. A therapeutically relevant level of hemoglobin is produced following administration of one or more of the presently disclosed transduced cells. Therapeutically relevant level of hemoglobin is a level of hemoglobin that is sufficient (1) to improve or correct anemia, (2) to restore the ability of the subject to produce red blood cells containing normal hemoglobin, (3) to correct ineffective erythropoiesis in the subject, (4) to correct extra-medullary hematopoiesis (e.g., splenic and hepatic extra- medullary hematopoiesis), and/or (5) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dL Hb, from about 11.5 g/dL Hb to about 12 g/dL Hb, from about 12 g/dL Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb.

In certain aspects, the therapeutically relevant level of hemoglobin is maintained in the subject for at least about 6 months, for at least about 12 months (or 1 year), for at least about 24 months (or 2 years). In certain aspects, the therapeutically relevant level of hemoglobin is maintained in the subject for up to about 6 months, for up to about 12 months (or 1 year), for up to about 24 months (or 2 years). In certain aspects, the therapeutically relevant level of hemoglobin is maintained in the subject for about 6 months, for about 12 months (or 1 year), for about 24 months (or 2 years). In certain aspects, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain aspects, the method comprises administering one or more cells transduced with a recombinant vector comprising a presently disclosed expression cassette as described above. The vector copy number of the recombinant vector in the cells that provide for the therapeutically relevant level of hemoglobin (e.g., 9-10 g/dL) in the subject is from about 0.5 to about 2, from about 0.5 to about 1, or from about 1 to about 2 vector copy number per cell. In certain aspects, the vector copy number of the presently disclosed vector is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 vector copy number per cell. The quantity of transduced cells to be administered will vary for the subject being treated. In certain aspects, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed transduced cells are administered to a subject. More effective cells may be administered in even smaller numbers. In certain aspects, at least about 1×10⁸ cells/kg, at least about 2×10⁸ cells/kg, at least about 3×10⁸ cells/kg, at least about 4×10⁸ cells/kg, or at least about 5×10⁸ cells/kg of the presently disclosed transduced cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain aspects, the cell is from a human leukocyte antigen (HLA)-matched donor. In certain aspects, the transduced cell is from the same subject. In certain aspects, the transduced cell is from bone marrow of the same subject. Thus, administration of the transduced cells does not incur the risk of graft-versus host disease in the subject. The method does not require immune suppression to prevent graft rejection, e.g., the method does not comprise administering an immunosuppressive agent to the subject.

### Kits

Also provided are kits comprising one or more of: the cells, expression cassettes, vectors, nuclease systems, nucleic acids and/or compositions of this disclosure and instructions for the manufacture of the same, and optionally, instructions for their therapeutic use as described herein. The kits find use in a variety of in vitro, ex vivo, and in vivo applications.

In certain aspects, provided are kits that include one or more of: cells, expression cassettes, vectors, nuclease systems and/or nucleic acids of the present disclosure, and instructions for introducing the expression cassette, vector, nuclease system and/or nucleic acid into a cell. The kits of the present disclosure may further include any other reagents useful for transfection/transduction, introducing the expression cassette, vector, nuclease system and/or nucleic acid into cells of interest.

These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, DVD, Bluray, computer readable memory device (e.g., a flash memory drive), etc., on which the information has been recorded. Yet another means that may be present is a website address which may be used via the Internet to access the information at a removed site. Any convenient means may be present in the kits.

The following examples are offered by way of illustration and not by way of limitation.

### EXPERIMENTAL

The present disclosure provides new components and methods to develop a new generation of highly efficient and safe gene therapy vectors for the treatment of hemoglobinopathies or other erythropoiesis disorders. The disclosure improves upon current viral vectors in terms of safety and efficiency while reducing the cost of these therapeutic approaches.

The increased efficiency and safety of these new generation therapy vectors is achieved by the addition of powerful but short erythroid enhancers. These enhancers were derived from DHS peaks that are only active during the erythroid development and differentiation and are absent in hematopoietic stem cells or other mature hematopoietic lineages. These new enhancers are almost ten times smaller in size compared to the μLCR, spanning a 300 bp region. Their short size can significantly improve lentiviral production yields, whereas erythroid specific activity ensures that these powerful elements will remain silent in other hematopoietic lineages.

These elements can be employed for the production of therapeutic lentiviral vectors for hemoglobinopathies to enhance the expression of the therapeutic transgene in a lineage specific manner. The following genes could be used in combination with the proposed enhancers: Beta-globin gene (HBB), Gamma-globin gene (HBG), shRNA targeting HbF repressors (BCL11a, LRF), homing endonucleases or DNA binding proteins aiming to reactivate the endogenous HbF expression.

These enhancers can achieve a higher expression of the transgene compared to a basal no enhancer vector, have an erythroid lineage specific activity and they can increase the titers of the produced vectors up to 5-fold compared to the μLCR.

### Example 1: Identification of Novel Enhancers

Experimental strategy. The experimental strategy employed was based on screening of the human genome for enhancers that can activate the beta globin gene promoter of a GFP gene expression cassette of a lentiviral vector, after transduction initially of an erythroid cell line and subsequently of primary human erythroid cells. Potential erythroid enhancers are sought among DNase hypersensitive sites (DHS) that are only active during the erythroid differentiation/maturation stages, but not in non-erythroid cell lineages.

Design and Synthesis of an erythroid specific DHS library. Putative enhancer elements where compiled from a list of developmentally regulated DNase I Hypersensitive Sites (DHS) during ex vivo induced erythropoiesis from mobilized adult human CD34+ cells. The initial list was filtered to exclude elements that were accessible in undifferentiated CD34+ cells. resulting in 5,710 DHS sequences. For each DHS, 198 bp-long overlapping sequences spanning the entire DHS (oligonucleotide sequences) were designed in such manner as to ensure that the core of DHS was represented by an entire oligo A total of 15,000 oligonucleotide sequences were designed and synthesized.

For the identification of the most potent erythroid specific enhancers a large number of HUDEP-2 cells (8×10⁶) was transduced with the new library at a low MOI (<0.4), to ensure for integration of not more than one vector copy per cell. The transductions were performed in three independent experiments. After 5 days of culture, the transduced cells were divided and sorted based on the intensity of the GFP expression into 5 distinct subpopulations: GFP−, GFP+, GFP-low, GFP-medium and GFP-high expressing cells. The DHS-inserts were amplified from genomic DNA (gDNA) isolated from these subpopulations and sequenced.

In order to identify elements with enhancing capacity the counts of each tile throughout the gates (GFP-low to GFP-high) were correlated with the MFI of each gate and selected for tiles with Pearson correlation (r)>0.9. The results were filtered further by excluding tiles with poor replication (r<0.7) and measured their enrichment score in the highest GFP gate compared to the total GFP population. This resulted in 307 tiles that map back to 198 DHSs. Several of these DHSs were synthesized, cloned into a lentiviral vector, produced as a single vector and compared side by side with positive controls (beta globin DHS2 and μLCR).

### Sequences of the New Enhancers:

SEQ ID NO: 1 ENH_PP:
ACATAATGATTTCCATTACGCTATTTCTGTGAAAT
GCAGCAGGTTCTTAAACGTTATTTCAGTGGCATGG
GCTGGAAGCTTATCACAAAAAGCCATGTGTGTGGC
CTTATCAGAACAGAAAGAGACAGGCTGGTGCCCAA
GGCTGCTGCCTGCTCCACCTTTTGCCAGCTCTGGA
CATCTGAGGACGTCCCGGCAGATCTGGAATGGGGC
CCTCAACTGACCATTTGCTTCTCAGAATTTCAGTT
TGAGACATGAGAGGTATAATCAGTTACTTTTCTCC
CCCCAGAGAAACCCTTTTGTG
SEQ ID NO: 2 ENH_SP:
TACCTATTTGCAAAAGAAGGGAAACCCCCGCGTAT
GGAACCAGAAGAGGGTTTCTTGCAGAAGGTGCCCC
AAGGTGTTGCAGTTTGATAACGCTCTCCTCATATC
TGTTAAAGAGCGCGGCAAATGTTATCTGGAAGCAG
CCGGGACAAGAGCACCCCCAGTTCTGAGCACAGTA
ACTGGGCTACTCAGAAGTTCCACTGAACGGCATGT
CAGTGCCTTACCAATAGTCACACTGAGCTGCTTGC
AAAGTAACTGCCACCACATTCCTTTCTTAAAGTGG
CCTGATTCCAAAGAGCTTGCCTAGAGCAAGGGGCA
GAAGAGCTGCCAAGTGCCTGCCATTTCCAGGAA
SEQ ID NO: 3 ENH_ST:
CCAGTTTACCACCTAAGAATAATAATAGTATCATC
TTCATAGGGGAGTTGTTGTGACTTTGAGATTAGCT
TTATCAAGTGTTTATAACAGTACCTGGCACATGGC
TGAATGAGTGCAACTCTGCCTGCTTTTGATTGAAG
TCATATTTTGTGACAGTTTCTGTTGAAGACTGGGC
ACCAAGCTAGACCACTTCATGTGCATTTTGGAGAT
AAAAGTTTTATCTAGCATTGCAGCTGCAAAGCCCC
TCCTCTCTCTCTTCACTTTGTTGGTTTCCTTTTTA
TCTTTACTTCGGTGTTTTTCAT
SEQ ID NO: 4 ENH_GC:
TCCCATCTATCTGCCTTGCATGGTGACAGCCAGCC
TTTAGCAACTTCTAAAAGAAAGAGGATCTATCAGC
TCAGTCTGAGCAGGCATGATAAGGTCTCCTCCCTT
GCCTGGAGGCACTCATCTGAGCCTCCAAACCACCC
CATTAGTGAATTCTCAGAAAGAACATTCTGCTGAG
AAAGAGGAGGATAGGTTCCCAGTCAAGGCTGGGAG
CTTGTAGCCACTAATCTCTTGGTGTCTAGATCAGG
TACAAGGCTGTCTCTCTGTCCCCACCCTCTCTGGT
CCCCTCCTCTTACTGTCCACT
SEQ ID NO: 5 ENH_BL:
GGGTGTGTGGCTCCTTAAGGTGACCCAGCAGCCCT
GGGCACAGAAGTGGTGCGTGGAGATAATGCCAACA
GTGATAACCAGCAGGGCCTGTCAGAAGAGGCCCTG
GACACTGAAGGCTGGGCACAGCCTTGGGGACCGCT
CACAGGACATGCAGCAGTGTGTGCCGACAACTCCC
TACCGCGACCCCTATCAGTGCCGACCAAGCACACA
AGATGCACACCCAGGCTGGGCTGGACAGAGGGGTC
CCACAAGATCACAGGGTGTGCCCTGAGAAGGTGGG
GAGCTCACAGCCTCCAAGCATTGCATCATCCTGGT
ACCAGGAAGGCAATGGGCTGCCCCATACCCACTTC
CCTTCCTAGAATTGGCCTGGGACA
SEQ ID NO: 6 ENH_GT:
TCCAAGGCAGCATGGCGGGCAAGAAGTTGAGGCCA
CTGTCCCTGGGTGTTCCTACCCCCACACCCTCACC
CCAAGACAGCCTGTTACTGCGGCGCCAACAGCCAC
GGTCGCCTACATCTGATAAGACTTATCTGCTGCCC
CAGGGCAGGCCGGAGCTGGCGTAAGCCCCAGTGGG
GCGCTAAGTGAGTGTGCCCCTGCCTCCCGCCAGCA
CTGGCCTGGCCTGCAGGCTTAGCCTGGGTCATCAA
GGTATCCCACAGGCTCTAGTTCAAATCCAGCAGAA
CCTCTCTGAGCCTCACTCTTC
SEQ ID NO: 7 ENH_DPR:
CAACAAAAACAAGCAAACAAATAGAACCTCAGGCT
CACGGCTGGGCAAGAGAGAAAGCACACGATGATGG
ACATCTGGAGCTTCCAGCAATGCATGGGCAGCAAA
GATAAGCTTTACTTGACTGCTGGGTAGGAGCACCA
GCAGAATGAAGCATAGACTATTTACCACACCCTAC
TTTGGCTTGGGCTAATAGTAAGTTACGCTTGTACA
AGGTCTTGGAAAGCAGCCAGTGCACTGGCACTCTA
AGCCTCACAAAAGATAGTGCTTTTCAGGTAGAAAA
TATATTCCACAATTGGTCTCT
SEQ ID NO: 8 ENH_PV1:
GTCCCAGGTCACACAGCTTGAGGGTGGCAGAAGCC
AGATCTCAGGTCACAGAGTGGCTCTCCTCCATTTG
CCTCCCTGGTCTTGCCTCCTGGAGGCCTGTAGATA
GGAGACACCCTTCCTGGAAAGACAACAGCTTGGGG
ACATTACAGATAAGGCTGGGCGGGTGTGCCTAGCT
AGCCCAGAATAAAAGGATCACCGAGAGAAGTTAGG
AATCGACCTTCCTTGAGGTGGGTTCTGCAGATAAA
GATGCTTTTGAAGTTCAGTTGGTGGGAGATAGGAT
CTTTGTGGCCTTCTGGATGGA
SEQ ID N0: 9 ENH_PV2:
CCAGTGGTTTGGGGACATGCTTCCTGGAGTCTTAT
CCCCTCTTGTCAGATTCAGGTTTAGGACAACGGTA
CAGTTGTTCCATTGTTCCCAGATAACATATCCTAT
CTTAACGTCTTTGCTGGGGCAGCCCTTGGACACAC
CCTGAGCTCCTGCCCTATCACTGGCCCCACTTGTA
CCCCTTGTGGGCAGGCACCACGGCAGCCTGCTCTG
AAGGGAGCTTTCATGTGGCCGACCAACTCTACTCC
TTGACTCCTCCCTTCTCTCATTGAAAAAAAATGTT
TAAGGGGACATTTCTCTCTCC
SEQ ID NO: 10 ENH_NP:
GTAGAAAGGGATCCTTTAAGATGAAATCGATGCCT
CCTCATGGAGTTCTAAGCCCTCAGAAGTGGAAGTC
AACTGGAGTGTGCAATTTTCTATCTTTCAACTAAC
CCAACAGTTTCTACCTCACCCTCCTGCCTTCCTAG
GACTCTGTGCACACACCCACCAGAGTGTTATCACA
TTGCAAGACAGAACCCAATTGTCTCCTTTATCTCC
TCTACCAGACTTTGTGCTCCATCAGTTGAAGAGTA
GGATGAAGAAAAAAAAAAAAGGTTACATGAAGATG
ATGGAATTATAATCAGGCCTT
SEQ ID NO: 11 ENH_PV1 + PV2:
GTCCCAGGTCACACAGCTTGAGGGTGGCAGAAGCC
AGATCTCAGGTCACAGAGTGGCTCTCCTCCATTTG
CCTCCCTGGTCTTGCCTCCTGGAGGCCTGTAGATA
GGAGACACCCTTCCTGGAAAGACAACAGCTTGGGG
ACATTACAGATAAGGCTGGGCGGGTGTGCCTAGCT
AGCCCAGAATAAAAGGATCACCGAGAGAAGTTAGG
AATCGACCTTCCTTGAGGTGGGTTCTGCAGATAAA
GATGCTTTTGAAGTTCAGTTGGTGGGAGATAGGAT
CTTTGTGGCCTTCTGGATGGANNNNNNNNNNCCAG
TGGTTTGGGGACATGCTTCCTGGAGTCTTATCCCC
TCTTGTCAGATTCAGGTTTAGGACAACGGTACAGT
TGTTCCATTGTTCCCAGATAACATATCCTATCTTA
ACGTCTTTGCTGGGGCAGCCCTTGGACACACCCTG
AGCTCCTGCCCTATCACTGGCCCCACTTGTACCCC
TTGTGGGCAGGCACCACGGCAGCCTGCTCTGAAGG
GAGCTTTCATGTGGCCGACCAACTCTACTCCTTGA
CTCCTCCCTTCTCTCATTGAAAAAAAATGTTTAAG
GGGACATTTCTCTCTCC

TABLE 1
Vectors comprising the new enhancers:

| Vector Name | Enhancer (SEQ ID NO) |
|---|---|
| L070 | ENH_PP (SEQ ID NO: 1) |
| L068 | ENH_SP (SEQ ID NO: 2) |
| L077 | ENH_ST (SEQ ID NO: 3) |
| L065 | ENH_GC (SEQ ID NO: 4) |
| L067 | ENH_BL (SEQ ID NO: 5) |
| L074 | ENH_GT (SEQ ID NO: 6) |
| L079 | ENH_DPR (SEQ ID NO: 7) |
| L178 | ENH_PV1 (SEQ ID NO: 8) |
| L177 | ENH_PV2 (SEQ ID NO: 9) |
| L069 | ENH_NP (SEQ ID NO: 10) |
| L177 + L178 | ENH_PV1 + PV2 (SEQ ID NO: 11) |

Notwithstanding the appended claims, the present disclosure is further defined by the following aspects:

1. An expression cassette comprising at least one copy of an enhancer element, wherein the enhancer element comprises a nucleotide sequence at least 50% identical to any one of SEQ ID NOs: 1-10.

2. The expression cassette of aspect 1, wherein the enhancer element comprises an erythroid transcription factor binding site, wherein the erythroid transcription factor is selected from the group consisting of GATA1, TAL1 and KLF1.

3. The expression cassette of aspect 1 or aspect 2, wherein the expression cassette comprises multiple copies of the enhancer element.

4. The expression cassette of aspect 3, wherein the expression cassette comprises two copies of the same enhancer element.

5. The expression cassette of aspect 3, wherein the expression cassette comprises a first enhancer element and a second enhancer element having different sequences, wherein optionally the first enhancer element comprises the sequence set forth in SEQ ID NO: 9 and the second enhancer element comprises the sequence set forth in SEQ ID NO: 8 or wherein optionally the first enhancer element comprises the sequence set forth in SEQ ID NO: 8 and the second enhancer element comprises the sequence set forth in SEQ ID NO: 9.

6. The expression cassette of any one of aspects 1 to 5, further comprising a sequence encoding a therapeutic agent.

7. The expression cassette of aspect 6, wherein the therapeutic agent comprises a polynucleotide or a polypeptide.

8. The expression cassette of aspect 6 or aspect 7, wherein the sequence comprises a globin gene.

9. The expression cassette of aspect 8, wherein the therapeutic agent comprises a globin polypeptide, wherein the globin polypeptide is a β-globin, a γ-globin, or a δ-globin.

10. The expression cassette of aspect 8 or aspect 9, wherein the globin gene is operably linked to an erythroid specific promoter.

11. The expression cassette of any one of aspects 1 to 10, wherein the erythroid specific promoter is a beta-globin promoter, an erythroid lineage-specific glycophorin A, Ankyrin, beta-Spectrin or Adducine 2 promoter.

12. A vector comprising the expression cassette of any one of aspects 1 to 11.

13. The vector of aspect 12, wherein the vector is a retroviral vector or a lentiviral vector.

14. The vector of aspect 12 or aspect 13, further comprising one or more of the following:

(a) a restriction site,

(b) an untranslated region,

(c) a DNase1-hypersensitive site,

(d) a multiple cloning site,

(e) a long terminal repeat, and

(f) a sequence encoding a poly A tail.

15. The vector of any one of aspects 12 to 14, comprising a first enhancer element, a promoter, a therapeutic gene, and a second enhancer element.

16. A cell transduced with the expression cassette of any one of aspects 1 to 11.

17. A cell transduced with the vector of any one of aspects 12 to 15.

18. The cell of aspect 16 or aspect 17, wherein the cell is selected from the group consisting of a hematopoietic stem cell, an embryonic stem cell, an induced pluripotent stem cell, and a hemogenic endothelium cell.

19. The cell of aspect 18, wherein the hematopoietic stem cell is a CD34+ hematopoietic stem cell.

20. The cell of any one of aspects 16 to 19, wherein the cell is transduced ex vivo.

21. A pharmaceutical composition comprising an effective amount of one or more of: the cell of any one of aspects 16 to 20, the expression cassette of any one of aspects 1 to 11, the vector of any one of aspects 12 to 15; and a pharmaceutically acceptable carrier.

22. A method of treating a hemoglobinopathy in a subject, comprising administering an effective amount of the pharmacological composition of aspect 21 to the subject.

23. The method of aspect 22, wherein the hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease.

24. The method of aspect 23, wherein the hemoglobinopathy is β-thalassemia.

25. The method of aspect 23, wherein the hemoglobinopathy is sickle cell anemia.

26. The method of any one of aspects 22 to 25, wherein the subject is a human.

27. The method of any one of aspects 22 to 26, wherein the cell is from the subject.

28. The method of aspect 27, wherein the cell is from bone marrow of the subject.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein.

1. An expression cassette comprising at least one copy of an enhancer element, wherein the enhancer element comprises a nucleotide sequence at least 50% identical to the nucleotide sequence set forth in any one of SEQ ID NOs: 1-10.

2. The expression cassette of claim 1, wherein the enhancer element comprises an erythroid transcription factor binding site, wherein the erythroid transcription factor is selected from the group consisting of GATA1, TAL1 and KLF1.

3. The expression cassette of claim 1, wherein the expression cassette comprises multiple copies of the enhancer element.

4. The expression cassette of claim 3, wherein the expression cassette comprises two copies of the same enhancer element.

5. The expression cassette of claim 3, wherein the expression cassette comprises a first enhancer element and a second enhancer element having different sequences.

6. The expression cassette of claim 1, further comprising a sequence encoding a therapeutic agent.

7. (canceled)

8. The expression cassette of claim 6, wherein the sequence encoding a therapeutic agent comprises a globin gene.

9. The expression cassette of claim 8, wherein the therapeutic agent comprises a globin polypeptide, wherein the globin polypeptide is a β-globin, a γ-globin, or a δ-globin.

10. The expression cassette of claim 8, wherein the globin gene is operably linked to a human erythroid promoter.

11. A vector comprising the expression cassette of claim 1, wherein the vector is a retroviral vector or a lentiviral vector.

12. (canceled)

13. The vector of claim 11, further comprising one or more of the following:
(a) a restriction site,
(b) an untranslated region,
(c) a DNaseI-hypersensitive site,
(d) a multiple cloning site,
(e) a long terminal repeat, and
(f) a sequence encoding a poly A tail, and/or
comprising a first enhancer element, a promoter, a therapeutic gene, and a second enhancer element.

14. (canceled)

15. The vector of claim 11, further comprising a cytomegalovirus (CMV) promoter, Rous sarcoma virus (RSV) promoter, simian virus 40 (SV40) promoter, or mammalian elongation factor 1α (EF1α) promoter.

16. A cell comprising the expression cassette of claim 1.

17. (canceled)

18. The cell of claim 16, wherein the cell is selected from the group consisting of a hematopoietic stem cell, an embryonic stem cell, an induced pluripotent stem cell, and a hemogenic endothelium cell.

19. The cell of claim 18, wherein the hematopoietic stem cell is a CD34+ hematopoietic stem cell.

20. (canceled)

21. A pharmaceutical composition comprising an effective amount of the cell of claim 16; and a pharmaceutically acceptable carrier.

22. A method of treating a hemoglobinopathy in a subject, comprising administering an effective amount of the pharmacological composition of claim 21 to the subject.

23. The method of claim 22, wherein the hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease.

24.-25. (canceled)

26. The method of claim 22, wherein the subject is a human.

27. The method of claim 26, wherein the cell is from the subject.

28. (canceled)
